(19) United States
(12) Patent Application Publication
(10) Pub. No.: US 2009/0264760 A1
(43) Pub. Date: Oct. 22, 2009

(54) COMPOUNDING IN MEDICAL DIAGNOSTIC ULTRASOUND FOR INFANT OR ADAPTIVE IMAGING

(75) Inventors: Roee Lazebnik, Palo Alto, CA (US); Richard Chiao, Cupertino, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL SOLUTIONS USA, INC., Malvern, PA (US)

(21) Appl. No.: 12/182,963

(22) Filed: Jul. 30, 2008

Related U.S. Application Data
(60) Provisional application No. 61/125,028, filed on Apr. 21, 2008.

(51) Int. Cl. A61B 8/13

(52) U.S. Cl. 600/447; 382/128

(57) ABSTRACT

Information is compounded in medical diagnostic ultrasound. Volumes from multiple acoustic windows for the infant head are aligned and combined. The combination provides a dataset better representing the entire region of interest. Additionally or alternatively, weighted combination of ultrasound data sets is provided. The weights adapt as a function of proximity to the transducer (e.g., near field verses far field), noise level, or other data quality parameters. In the infant head example, the adaptive weights may provide a composite data set better representing the infant head. Adaptive weights for the compounding may be used in situations other than an infant head scan.

### RELATED APPLICATIONS

The present patent document claims the benefit of the filing date under 35 U.S.C. §119(e) of Provisional U.S. patent application Ser. No. 61/125,028, filed Apr. 21, 2008, which is hereby incorporated by reference.

### BACKGROUND

The present embodiments relate to compounding in medical diagnostic ultrasound, such as compounding scans of an infant head. Cranial sonographic evaluation may be used to image intracranial hemorrhage (ICH), ischemic brain injury, vascular malformations, and anatomic abnormalities.

Modern sonographic examination of newborns is typically performed with a sector transducer using 5 MHz or higher frequency. Standard images are acquired through three acoustic windows: anterior or frontal fontanel (AF), posterior or occipital fontanel (PF), and the mastoid fontanel (MF). Each of these acoustic windows is more optimal for a different subset of anatomy to be evaluated. The AF, which closes by age nine to 15 months, is typically the primary imaging window. The PF may provide improved sensitivity for identifying an intraventricular hemorrhage, particularly when the lateral ventricles are not dilated. The PF may allow improved visualization of the occipital periventricular white matter, an area of particular concern in periventricular leukomalacia. The PF and MF may be particularly useful in evaluating the posterior fossa for hemorrhage or anomalies, such as Chiari malformation.

The typical two-dimensional sonographic exam generates at least six coronal images (at the levels of the frontal horns anterior to the foramen of Monro, thalami, quadrigeminal plate cistern, atria of lateral ventricles, and posterior to the lateral ventricles) and five sagittal images (midline and two parasagittal views on each side). These images represent planes within the infant head. An axial plane demonstrating the cerebellum is often obtained using the MF window. However, the limited accessibility to ultrasound (i.e., the acoustic window locations) may not allow scanning the desired planes. The transducer must be rocked or rotated to acquire images representing non-parallel planes.

Volume acquisition of sonographic data may be used. The transducer is positioned at one of the fontanels, and a volume scan is performed. Planes that are diagnostically useful but cannot be directly acquired by two-dimensional scans may be reconstructed from volume data. Anatomy or pathology may be more easily examined across continuous spatial regions to better localize and delineate features. Volumetric quantitative measurements may be possible instead of two-dimensional approximations.

Volume acquisition however does not circumvent some limitations of different acoustic windows. The position and size of the acoustic windows may limit the volume available for scanning. Due to distance from an acoustic window and the high imaging frequency, some far field portions of the images may have poor quality. The data may have differing degrees of data quality for a given anatomical region. Present methods for diagnostic evaluation use manual evaluation of several independent volumes to minimize these problems.

### BRIEF SUMMARY

By way of introduction, the preferred embodiments described below include a method, system, instructions, and computer readable media for compounding information in medical diagnostic ultrasound. Volumes from multiple acoustic windows for the infant head are aligned and combined. The combination provides a dataset better representing the entire region of interest. Additionally or alternatively, weighted combination of ultrasound data sets is provided. The weights adapt as a function of proximity to the transducer (e.g., near field verses far field), noise level, or other data quality parameters. In the infant head example, the adaptive weights may provide a composite data set better representing the infant head. Adaptive weights for the compounding may be used in situations other than an infant head scan.

In a first aspect, a method is provided for compounding infant head information in medical diagnostic ultrasound. An infant head is scanned from first and second acoustic windows with ultrasound. First and second data responsive to the scans from the first and second acoustic windows, respectively, are spatially registered. The first and second data are combined as a function of the spatial registering.

In a second aspect, a system for compounding head information in medical diagnostic ultrasound is provided. An ultrasound imaging system is operable to scan an internal region of a patient with the transducer positioned adjacent to different fontanels. A processor is operable to combine data from scans for the different fontanels into a composite data set and output image data as a function of the composite data set. A display is operable to generate an image as a function of the output image data.

In a third aspect, a computer readable storage medium has stored therein data representing instructions executable by a programmed processor for compounding infant head information in medical diagnostic ultrasound. The storage medium includes instructions for registering data from multiple slice or volume infant head sonographic acquisitions corresponding to different fontanels, and assembling a dataset representing a volume from the registered data.

In a fourth aspect, a method is provided for compounding information in medical diagnostic ultrasound. First and second sets of data representing first and second overlapping regions, respectively, are spatially registered. For each spatial location, a relative weighting is determined as a function of proximity to a transducer during acquisition, noise, or combinations thereof. The first and second sets of data are compounded as a function of the relative weightings.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of an ultrasound system for compounding head information in medical diagnostic ultrasound;

FIG. 2 is a graphical representation of an example infant head showing fontanels;

FIGS. 3A and 3B show graphical representations of planar images from a component scan and a composite dataset; and

FIG. 4 is a flow chart diagram of one embodiment of a method for compounding head information in medical diagnostic ultrasound.

### DETAILED DESCRIPTION OF THE DRAWINGS AND PRESENTLY PREFERRED EMBODIMENTS

Neonatal (or older baby) head sonography typically uses multiple acquisitions through different acoustic windows (frontal fontanel, posterior fontanel, and mastoid fontanel) to optimally visualize intracranial structures. These individual data set acquisitions are each optimal for different regions of anatomy. Using flexible “shape tape,” magnetic position tracking, data correlation or other alignment for automatic spatial localization of transducer positions, multiple slices or volume acquisitions at different fontanels may be co-registered. The registered data sets may be combined to form a single intracranial data volume. Within this volume, each anatomical structure may be visualized using data from a single or spatially compounded combination of the most optimal acquired data sets. Multiple reformatted images of arbitrary orientation may be extracted from this volume.

Multiple slice or volume neonatal head sonographic acquisitions are automatically registered, such as using transducer localization to determine relative scan positions. Data representing a composite volume is automatically assembled through spatial compounding of acquired data. The combination may be based on expected or computed quality of data for each acquisition volume region.

Volumetric sonographic data is automatically assembled for evaluation of neonatal or infant intracranial contents. 2D planes or volumes from multiple trans-fontanel acquisitions are combined. Each acquisition is assigned a spatial location based on a transducer's position or spatial locations represented by data. For example, a flexible localization strip (“shape tape”) is applied to the patient's head (e.g., 1^{st} vertebrae). Using spatial information from the localization strip and/or the sonographic data, the composite volume of sonographic data is generated.

This composite volume may account for the data quality from each of the contributing acquisitions through automated image or volume evaluation and/or heuristic knowledge. The composite volume may represent spatially compounded sonographic data such that for a given voxel, contributors are weighted to optimize that voxel's image quality.

There are multiple diagnostic applications for neonatal head imaging, including intracranial bleed detection/quantification, brain parenchyma evaluation, congenital anomaly diagnosis, 3D vascular assessment, and others. Diagnostic confidence and/or workflow efficiency in neonatal head evaluation may be improved by automated compositing and quality weighting in the compositing of data acquired from different acoustic windows.

FIG. 1 shows a system 10 for compounding head information in medical diagnostic ultrasound. The system 10 includes a transducer 12, a location device 14, a reference sensor 16, an ultrasound imaging system 18, a processor 20, a memory 22, and a display 24. Additional, different, or fewer components may be provided. For example, the system 10 does not include the location device 14 and/or the reference device 14. As another example, the system 10 includes a user interface. In one embodiment, the system 10 is a medical diagnostic ultrasound imaging system. In other embodiments, the processor 20 and/or memory 22 are part of a workstation or computer different or separate from the ultrasound imaging system 18. The workstation is adjacent to or remote from the ultrasound imaging system 18.

The transducer 12 is a single element transducer, a linear array, a curved linear array, a phased array, a 1.5 dimensional array, a two-dimensional array, a radial array, an annular array, a multidimensional array, a wobbler, or other now known or later developed array of elements. The elements are piezoelectric or capacitive materials or structures. In one embodiment, the transducer 12 is adapted for use external to the patient, such as including a hand held housing or a housing for mounting to an external structure.

The transducer 12 converts between electrical signals and acoustic energy for scanning a region of the patient body. The region of the body scanned is a function of the type of transducer array and position of the transducer 12 relative to the patient. For example, a linear transducer array may scan a rectangular or square, planar region of the body. As another example, a curved linear array may scan a pie shaped region of the body. Scans conforming to other geometrical regions or shapes within the body may be used, such as Vector® scans. The scans are of a two-dimensional plane. Different planes may be scanned by moving the transducer 12, such as by rotation, rocking, and/or translation. Alternatively, a volume is scanned. The volume is scanned by electronic steering alone (e.g., volume scan with a two-dimensional array), or mechanical and electrical steering (e.g., a wobbler array or movement of an array for planar scanning to scan different planes).

Optionally, the transducer 12 includes the location device 14. The location device 14 is in or on the ultrasound transducer 12. For example, the location device 14 is mounted on, placed within, or formed as part of the housing of the transducer 12. Signals or data are provided from or to the location device 14 with wires in the transducer cable or wirelessly.

The location device 14 is a sensor or sensed object. For example, the location device 14 includes coils of a magnetic position sensor. The coils sense a magnetic field generated by another device external to the sensor. Alternatively, the magnetic field is generated by the location device 14 and coils spaced from the location device 14 sense the position information of the transmitter.

The location device 14 may be part of a magnetic position sensor. Three orthogonal coils are provided. By sequencing transmission through remote transmitter coils and measuring signals on each of the sensors coils, the location and orientation of the sensor coil is determined. Based on the position and orientation of the patient relative to the transmitter coils, the location and orientation of the transducer 12 is determined.

Other location devices 14 may be used. For example, a gravity sensor indicates the orientation of the transducer relative to the center of the earth. In other examples, the location device 14 is an accelerometer or gyroscope. An optical sensor may be used, such as the location device 14 being a pattern, light transmitter, or the housing of the transducer 12. A camera images the transducer 12. A processor determines the orientation and/or position based on the location in the field of view, distortion, and/or size of the location device 14.

Other orientation sensors may be used for sensing one, two or three degrees of orientation relative to a reference. Other position sensors may be used with one, two or three degrees of position sensing. In other embodiments, a position and orientation sensor provide up to 6-degrees of position and orientation information. Examples of magnetic position sensors that offer the 6 degrees of position information are the Ascension Flock of Birds and the Biosense Webster position-sensing catheters.

In another embodiment, the location device 14 is a fiber optic position sensor, such as the Shapetape sensor available from Measurand, Inc. The orientation and/or position of one end or portion of the fiber optic position sensor relative to another end or portion are determined by measuring light in fiber optic strands. One end or other portion of the fiber optic position sensor is held adjacent to a known location, such as the first vertebrae. The bending, twisting, and rotation of the fiber optic positions sensor is measured, such as measuring at a time after the transducer is positioned adjacent an acoustic window. The relative position of the transducer at different acoustic windows may be determined.

The reference sensor 16 is also a location device. In one embodiment, the same type of location device as the location device 14 is used. Frequency, coding, timing or other characteristics allow separate position and/or orientation sensing of the reference sensor 16 and the location device 14. In other embodiments, the reference sensor 16 and the location device 14 are different types of devices.

The reference sensor 16 is a wireless or wired device for providing location information or receiving transmit information from the processor 20 or another device. The reference sensor 16 is positionable in a known location, such as over the sternum, at a left or right shoulder, or at the navel of the patient. Glue or other sticky material may maintain the reference sensor 16 in place.

The ultrasound imaging system 18 is a medical diagnostic ultrasound system. For example, the ultrasound imaging system 18 includes a transmit beamformer, a receive beamformer, a detector (e.g., B-mode and/or Doppler), a scan converter, and the display 24 or a different display. The ultrasound imaging system 18 connects with the transducer 12, such as through a releasable connector. Transmit signals are generated and provided to the transducer 12. Responsive electrical signals are received from the transducer 12 and processed by the ultrasound imaging system 18. The ultrasound imaging system 18 causes a scan of an internal region of a patient with the transducer 12 and generates data representing the region as a function of the scanning. The data is beamformer channel data, beamformed data, detected data, scan converted data, and/or image data. The data represents anatomy of the region, such as the interior of an infant head, the heart, liver, fetus, muscle, tissue, fluid, or other anatomy.

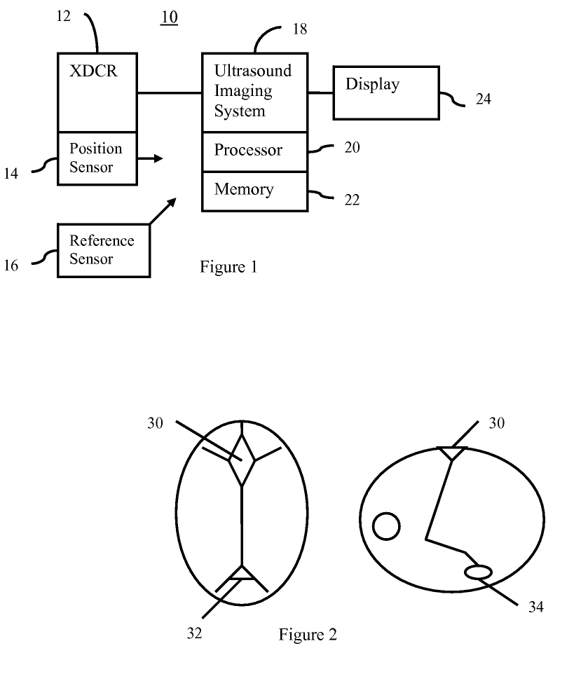

The ultrasound imaging system 18, using the transducer 12, may acquire multiple data sets. For example, one or more scans of a same region are performed with the transducer 12 at each of at least two different fontanels. One or more two-dimensional planes are scanned at each fontanel. Alternatively or additionally, a volume is scanned at each fontanel. FIG. 2 shows a top and side view graphical representation of an infant head having a frontal fontanel 30, occipital fontanel 32, and a mastoid fontanel 34. A scan is performed at two or more of the fontanels 30, 32, 34.

In another embodiment, the ultrasound imaging system 18 is a workstation or computer for processing ultrasound data. Ultrasound data is acquired using an imaging system connected with the transducer 12 or using an integrated transducer 12 and imaging system. The data at any level of processing (e.g., radio frequency data (e.g., I/Q data), beamformed data, detected data, and/or scan converted data) is output or stored. For example, the data is output to a data archival system or output on a network to an adjacent or remote workstation. The ultrasound imaging system 18 processes the data further for analysis, diagnosis, and/or display.

The processor 20 is one or more general processors, digital signal processors, application specific integrated circuits, field programmable gate arrays, controllers, analog circuits, digital circuits, server, combinations thereof, network, or other logic devices for compounding data from different scans. A single device is used, but parallel or sequential distributed processing may be used. In one embodiment, the processor 20 is a system controller of the ultrasound imaging system 18. The processor 20 receives inputs from any location device 14 and any reference sensor 16.

The processor 20 combines data from scans for the different fontanels into a composite data set. To combine the data, the regions represented by the data sets are spatially registered. In one embodiment, cross-correlation, minimum sum of absolute differences, or other similarity function is used to identify the relative translation and/or orientation of the regions. The best or sufficient match of the data to each other is determined. The translation and/or rotation associated with the match indicate the different or relative positions of the regions represented by the data. The match spatially aligns the data from the scans for the different fontanels.

In another embodiment, the processor 20 receives spatial indications from the location sensor for aligning the regions represented by the data. The location device 14, with or without reference to the reference sensor 16, indicates a position of the transducer 12 during a given scan. Absolute or relative position information may be used. For scans from different fontanels, the processor 20 determines a spatial relationship of the scans from the position and orientation of the transducer 12 during the scans. The relationship information may be used to align the data from the scans from different fontanels.

Multiple sources of alignment information may be used. For example, both data-based and sensor-based relative positions and orientations are determined. Average position and orientation are used. One source may be used for position and another source may be used for orientation. One source may be used to assure that a primary source is correct.

Once aligned, the processor 20 is operable to combine the data. The data from different scans for the different fontanels are compounded as a function of the spatial alignment. Where data from multiple sets or different scans represents a same spatial location, the data is combined, such as averaged. Due to the different scan formats and/or different acoustic windows, the data may generally represent a same spatial location, but not exactly align. Data from one or more scans may be converted or formatted to a grid associated with another of the scans or a reference grid. For example, the data representing different volumes is interpolated to a three-dimensional reference grid. After conversion, values for data from multiple volumes are combined. Alternatively, a nearest neighbor or other approach is used to determine the data to be combined.

Since the scanned volumes may not be identical, different spatial locations may be associated with a different number of values to be combined. For example, one spatial location may be represented by a single value from the AF scan. Another spatial location may be represented by two values from scans from two of the fontanels. Another spatial location may be represented by three values, one from each of the fontanel scans. Normalized or averaged combination is used. Filtering may be provided to reduce any artifacts from combining different numbers of values for different spatial locations.

The values are combined by averaging. Other combination functions may be used, such as a maximum or minimum value selection. In one embodiment, a weighted average is used. The processor 20 weights the values prior to averaging. The weighting may be predetermined or fixed. For a simple average, the weights are set based on the number of contributing values.

In one embodiment, the weights adapt as a function of the spatial location, data quality, or combinations thereof. For example, near field or mid field information may be better quality than far field or very near field data. Data in the middle of a scan field may be better quality than data associated with larger steering angles. The better quality data is weighted more heavily. For example, near field data from an AF scan is weighted more heavily than far field data from a PF scan. Wobbler transducers may provide better quality information for one array orientation than another, such as due to speed of movement of the array. The better quality data may be weighted more heavily.

The data may be processed to determine the quality or a quality factor. For example, the noise level associated with different spatial locations is determined. The standard deviation in a generally homogenous region may indicate a level of noise for the scan or a portion of the scan. As another example, a measure of high frequency variation indicates the noise level. In another example, the magnitude of the return without time or depth gain compensation is compared to a threshold level or slope to determine a noise level as a function of depth. Noise levels may be determined for different portions of a scan. The noise at other locations in interpolated. The quality for a given value is indicated by the level of noise.

Any variance or difference in weighting may be used. The weighting is relative, such as all the weights adding to unity. A difference in quality between values may be determined and the relative weighting set based on the difference. For example, if two values have similar quality, then equal weighting is provided. If the two values have different quality, then unequal weighting is provided. One or more factors may be used to determine overall quality. The factors may be weighted differently depending on importance or reliability.

The processor 20 uses the composite volume for quantification, imaging, and/or archiving. The data of the composite volume may be segmented or border detection applied to determine volume values or isolate information associated with particular structures. The dataset representing the composite volume may be output as image data. The image data may be data at any stage of processing, such as prior to or after detection. The image data may be specifically formatted for display, such as red, green, blue (RGB) data. The image data may be prior to or after any mapping, such as gray scale or color mapping.

The memory 22 is a tape, magnetic, optical, hard drive, RAM, buffer or other memory. The memory 22 stores the data from the different scans and/or the data of the composite volume.

The memory 14 is additionally or alternatively a computer readable storage medium with processing instructions. Data representing instructions executable by the programmed processor 20 is provided for compounding infant head information in medical diagnostic ultrasound. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The display 24 is a CRT, LCD, projector, plasma, printer, or other display for displaying two-dimensional images or three-dimensional representations. The display 20 displays ultrasound images as a function of the output image data. For example, a multi-planar reconstruction (MPR) of two or more images representing orthogonal planes is provided. As another example, a plurality of ultrasound images representing two or more parallel planes in the internal region are provided, such as six or more coronal images, five or more sagittal images, an axial image, and/or combinations thereof. Volume or surface rendering may alternatively or additionally be used.

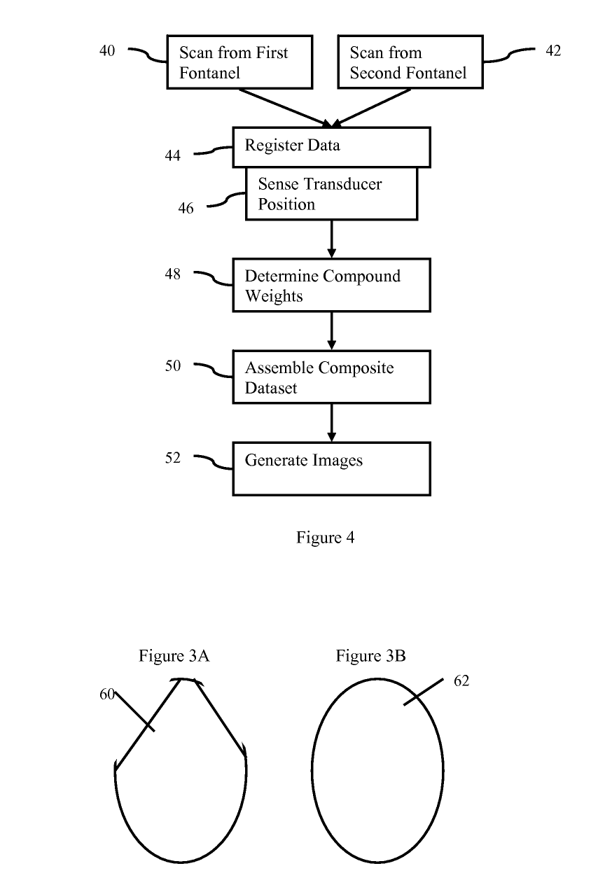

One or more of the images has a first portion responsive to data from a first of the different fontanels and not a second of the different fontanels. For example, FIG. 3A shows a scan region 60 from the AF for a plane. Due to the steering limits of the array and/or the size of the acoustic window, a portion of the cross-section of the infant head is not scanned. FIG. 3B shows a composite volume region at the same cross section. Due to far field data from the MF or PF, the missing portion of the cross-section is provided. The entire cross section may be imaged due to the compounding of volumes from different fontanels. There may be some locations for which no data is available. Due to any adaptive weighting, better quality information may be provided at different spatial locations.

FIG. 4 shows a method for compounding infant head information in medical diagnostic ultrasound. The acts of FIG. 4 are implemented by the system 10 of FIG. 1 or a different system. The acts shown in FIG. 4 are performed in the order shown or a different order. Additional, different, or fewer acts may be performed. For example, act 46 may not be used. As another example, act 48 is not provided.

In acts 40 and 42, an infant head is scanned with ultrasound from different acoustic windows. Any type of scanning may be used, such as planar or volume scanning. For planar scanning, multiple planes are sequentially scanned. The transducer may be rocked, rotated, translated or otherwise moved to scan the different planes from the same acoustic window. For example, perpendicular planes are scanned by rotation of the transducer or aperture. Alternatively, a single plane is scanned. The same region may be scanned multiple times from the same acoustic window. The resulting data is combined, such as by persistence filtering, or a more optimal one of the resulting data sets is selected.

The scanning may be for B-mode, color flow mode, tissue harmonic mode, contrast agent mode or other now known or later developed ultrasound imaging modes. Combinations of modes may be used, such as scanning for B-mode and Doppler mode data. Any ultrasound scan format may be used, such as a linear, sector, or Vector®. Using beamforming or other processes, data representing the scanned region is acquired.

The scanning is from different acoustic windows. Any two or more different acoustic windows may be used. The transducer is sequentially positioned at different windows. Alternatively, multiple transducers are used to allow either sequential or simultaneous scanning from different windows. For an infant or neo-natal head, the acoustic windows include the anterior fontanel, posterior fontanel, and mastoid fontanel. Other windows may be available due to surgery or deformity. One of the fontanels may have closed due to age of the infant. For non-infant head scans, various acoustic windows may be available depending on the anatomy being scanned. For imaging other portions of the body, other windows may be available.

In act 44, data from multiple acquisitions is registered. Since different acoustic windows are used, the regions represented by the data are aligned such that the values representing the same structure may be identified and combined. For example, multiple slice or volume infant head sonographic acquisitions from different fontanels are registered. The data for the acquisitions represents an overlapping region. Some data from each acquisition may represent locations not represented in another acquisition. The data sets are spatially registering to align the overlapping regions.

In one embodiment, the data is correlated to determine the alignment. In act 46, the position of the transducer is sensed for aligning. For example, a flexible fiber optic sensor (e.g., flexible localization strip such as shape tape) determines relative position and orientation along the flexible sensor. The location is along a line, in a plane, or in three dimensions. The flexible localization strip has electronic output that enables the spatial location of each ultrasound image voxel to be determined relative to the reference point. One end or portion of the flexible localization strip is positioned at a fixed or known location, such as attaching to a table or position on a patient (e.g., 1^{st} vertebrae), and the other end or portion of the flexible localization strip is connected to the transducer. Magnetic, optic, gravity sensor, accelerometer, gyroscope, optical pattern recognition, infrared, radio frequency, or other position sensors may alternatively indicate relative or absolute position.

The ultrasound imaging system determines the spatial relationship of the voxels or data samples to the transducer, and the transducer sensing provides the relative or absolute position of the transducer. This allows every voxel of each sonographic acquisition to be assigned a spatial position. During acquisition, the position and/or orientation of the transducer are sensed. The transducer position at each acoustic window is determined for spatially aligning the resulting acquisition data.

The position information is a position, orientation, or position and orientation of the transducer. The position information corresponds to one or more degrees of freedom or sensing, such as sensing 6 degrees of freedom (three translational axes and three rotational axes). The position information is sensed at the transducer or based on signals sent from the transducer.

In act 48, weights are determined for combining data from the aligned data sets. For each spatial location, a weighting of the contributing values is determined. By weighting one or more of the contributing values, a relative weight of the contributing values is determined. In one embodiment, the weighting is determined as a function of proximity to a transducer during acquisition, noise, or combinations thereof. Each voxel or data value is assigned a weighting based on the spatial location, such as relative to heuristic assumptions of anatomy and acquisition depth, and/or quality, such as determined through signal or image processing techniques. The assignment of relative weights may better optimize overall image quality for the composite dataset when multiple values with for the same spatial location are acquired from different sonographic acquisitions. For example, data acquired using the AF window is weighted such that voxels describing the posterior fossa (far field) are little weighted. In contrast, the same anatomical location voxels as visualized from the MF window (near field) are highly weighted. A greater weighting is assigned for near field data than far field data. As another example, a greater weighting is assigned for less noisy data than for more noisy data. The weights may include zero weighting or non-selection of a value to remove contribution of the value.

Other data quality factors may be used for determining the relative weighting. In other embodiments, the weights are predetermined or set, and/or equal weights are used.

In act 50, a dataset representing is assembled. A composite volume is assembled using the multiple sonographic acquisitions. The composite dataset represents a volume. The volume may be sampled by only two planes, such as two intersecting planes where data along the line of intersection are combined. The dataset may represent the combination of data from multiple planes, such as a plurality of planes acquired from each acoustic window. Each of the sonographic acquisitions is considered volumetric data even if composed of slice information since the slices from different windows may not be for a same plane. Alternatively, the volume is fully sampled or represents the combination of two volume scans.

The dataset is assembled from the registered data. The spatial relationships of the component datasets are determined. Using reformatting, a nearest neighbor selection, interpolation, or other process, values are determined for each spatial location represented in one or more of the component datasets.

Any combination function may be used, such as averaging. For a given voxel within the composite dataset, a combination of individual data elements is performed. Summation, multiplication, division, subtraction, or other functions may be used to determine a value for a spatial location from two or more values representing the spatial location.

In one embodiment, the sets of data are compounded as a function of the relative weightings determined in act 48. For example, a weighted spatial compounding technique is used. Each data element or value from the component datasets is weighted before averaging. Since the weights are assigned as a function of spatial location, data quality, or combinations thereof, spatial compounding is provided as a function of expected data quality, computed data quality, or both. When two or more values are combined to represent a spatial location, the value with the greater weighting is emphasized. For example, data associated with near field scanning is emphasized as compared to data associated with far field scanning. As another example, data associated with lower levels of noise is emphasized more than data associated with higher levels of noise. The values of the composite dataset more likely are associated with quality.

In act 52, one or more images are generated from the composite dataset. For example, a plurality of images representing parallel planes from the combined data is generated. Each of the plurality of images has an area larger than available from a single one of the scans alone. An image from any arbitrary plane may be generated from the composite data representing a volume. Alternatively, one or more two-dimensional images are generated along a scan plane. Due to compositing in act 50, the data along the scan plane may be responsive to different scans from different windows. In other embodiments, MPR, volume rendering, surface rendering, or other imaging is provided.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I(We) claim:

1. A method for compounding infant head information in medical diagnostic ultrasound, the method comprising:
scanning from a first acoustic window of an infant head with ultrasound;
scanning from a second acoustic window of the infant heat with ultrasound;
spatially registering first and second data responsive to the scans from the first and second acoustic windows, respectively; and
combining the first and second data as a function of the spatial registering.

2. The method of claim 1 wherein scanning from the first and second acoustic windows comprises volume scanning.

3. The method of claim 2 wherein the volume scanning comprises scanning along a plurality of different planes.

4. The method of claim 1 wherein scanning comprises scanning from at least two of an anterior fontanel, a posterior fontanel, and a mastoid fontanel.

5. The method of claim 1 further comprising generating a plurality of images representing parallel planes from the combined first and second data, each of the plurality of images having an area larger than available from the first and second data alone.

6. The method of claim 1 wherein spatially registering comprises correlating the first data with the second data.

7. The method of claim 1 wherein spatially registering comprises sensing a position of a transducer used for scanning form the first and second acoustic window.

8. The method of claim 7 wherein sensing the position comprises sensing with a magnetic sensor or sensing with a flexible strip operable to determine location and orientation from a known location.

9. The method of claim 1 wherein combining comprises weighted combining, weights being assigned as a function of spatial location, data quality, or combinations thereof.

10. The method of claim 9 wherein weighted combining comprises greater weighting for data associated with near field scanning as compared to data associated with far field scanning, greater weighting for data associated with lower levels of noise than data associated with higher levels of noise, or combinations thereof.

11. A system for compounding head information in medical diagnostic ultrasound, the system comprising:
a transducer;
an ultrasound imaging system operable to scan an internal region of a patient with the transducer positioned adjacent to different fontanels;
a processor operable to combine data from scans for the different fontanels into a composite data set and output image data as a function of the composite data set; and
a display operable to generate an image as a function of the output image data.

12. The system of claim 11 wherein the processor is operable to spatially register and compound the data from the different scans.

13. The system of claim 12 wherein the processor is operable to compound the data as a function of weights, the weights adaptive to the spatial location, data quality, or combinations thereof.

14. The system of claim 11 wherein the transducer has a location device, the combination of the data from the scans for the different fontanels being spatially aligned as a function of the location device.

15. The system of claim 14 wherein the location device comprises an optical imaging system, a magnetic position sensor, a fiber optic position sensor, or combinations thereof.

16. The system of claim 11 wherein the image comprises a plurality of ultrasound images representing two or more parallel planes in the internal region, at least one of the ultrasound images having a first portion responsive to data from a first of the different fontanels and not a second of the different fontanels and a second portion responsive to data from the second of the different fontanels.

17. In a computer readable storage medium having stored therein data representing instructions executable by a programmed processor for compounding infant head information in medical diagnostic ultrasound, the storage medium comprising instructions for:
registering data from multiple slice or volume infant head sonographic acquisitions corresponding to different fontanels; and
assembling a dataset representing a volume from the registered data.

18. The computer readable storage medium of claim 17 wherein registering comprises registering as a function of a position sensor connected with a transducer.

19. The computer readable storage medium of claim 17 wherein assembling comprises spatially compounding as a function of expected data quality, computed data quality, or both.

20. A method for compounding information in medical diagnostic ultrasound, the method comprising:
spatially registering first and second sets of data representing first and second overlapping regions, respectively;
for each spatial location, determining a relative weighting as a function of proximity to a transducer during acquisition, noise, or combinations thereof; and
compounding the first and second sets of data as a function of the relative weightings.

21. The method of claim 20 wherein spatially registering comprises sensing a position of the transducer used for acquisition of the first and second data sets, the first and second sets of data corresponding to scans from different locations.

22. The method of claim 20 wherein determining the relative weighting comprises assigning a greater weighting for near field data than far field data.

23. The method of claim 20 wherein determining the relative weighting comprises assigning a greater weighting for less noisy data than for more noisy data.
